(19) United States
(12) Patent Application Publication
(10) Pub. No.: US 2011/0212177 A1
(43) Pub. Date: Sep. 1, 2011

(54) CONFIGURABLE MICROSCOPIC MEDICAL PAYLOAD DELIVERY DEVICE TO DELIVER MEDICALLY THERAPEUTIC PAYLOADS TO SPECIFICALLY TARGETED CELL TYPES

(76) Inventors: Lane Bernard Scheiber, Annandale, VA (US); Lane Bernard Scheiber, II, Grosse Ile, MI (US)

(21) Appl. No.: 12/715,362

(22) Filed: Mar. 1, 2010

(51) Int. Cl. A61K 9/14; C07K 14/435

(52) U.S. Cl. 424/484; 530/350; 530/395

(57) ABSTRACT

The innovative strategy of treatment described here utilizes configurable microscopic medical payload delivery devices to act as a transport mechanism to deliver medically therapeutic payloads to specific cell types in the body. Utilizing probes on the exterior of the configurable microscopic medical payload delivery devices, these transport devices locate specific types of target cells in the body. Once a specific target cell is encountered and engaged, the configurable microscopic medical payload delivery device inserts its payload into the target cell. These medically therapeutic payloads are intended to improve cell function or the longevity of the cell or eliminate cells that pose a hazard to the general health of the body. By utilizing configurable microscopic medical payload delivery devices as a delivery system, the efficacy of medications and biologic tools are dramatically improved and there is a resultant significant reduction in the occurrence of unwanted side effects.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2010 Lane B. Scheiber II and Lane B. Scheiber. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to correct a deficiency in the body utilizing a configurable microscopic medical payload delivery device to transport and deliver a medical treatment payload into one or more specific cell types in the body.

2. Description of Background Art

Present medical care is attempting to utilize viruses to deliver genetic information into cells. Research in the field of gene therapy has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the exterior surface of a virus virion dictates which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA based payload to the cell type the viral vector naturally targets as its host cell.

Naturally occurring viruses also have the disadvantage of being susceptible to detection and elimination by a body's immune system. Viruses have been infecting humans for hundreds of thousands of years. A human's innate immune system is very efficient at detecting the presence of most naturally occurring viruses when such a virus is inside the body. The human immune system is quite capable of generating a vigorous response to most intruding viruses by attacking and neutralizing the virus virions whenever a viral virion physically exits outside the exterior wall of the virus's host cell. If gene therapy in its current state were to become a clinical therapeutic tool, the naturally occurring viruses selected for gene therapy research will have limited effectiveness due the fact that once the viral vector is introduced into the body, the body's immune system will quickly engage and eliminate the viral vectors, possibly before the vector is able to deliver its payload to its host cell or target cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

Cichutek does not claim the use of specific glycogen probes to target specific types of cells. Cichutek's approach is dependent upon the probes naturally present on the viral vectors reported in the patent, which will direct the viral vectors to only those cells the viruses naturally use as their host cell. Cichutek's approach is very restrictive: limited to gene transfer to only cells the viruses use as their natural host cell.

It is questionable that Cichutek's approach as described in the specification and claims is feasible. Cichutek's claim 4, states ‘The SIVagm vector of claim 1, wherein the viral envelope further comprises a single chain antibody (scFv) or a ligand of a cell surface molecule.’ By use of the words ‘a’ and ‘or’ in the claim, the claim is limited in the singular, meaning Cichutek claims a single chain antibody or a singular ligand. Singular type antibodies or ligands can be used for cell to cell communication, but to open an access portal into a cell and insert a payload into the cell requires two different types of antibodies or ligands. As an example, human immunodeficiency virus requires the use of both the gp120 and gp41 probes to open a portal into a T-Helper cell and insert its genome into the T-Helper cell. The gp120 probe engages the CD4+ cell-surface receptor on the T-cell. Once the gp120 probe has successfully engaged a CD4+ cell-surface receptor on the target T-Helper cell, then the HIV virion's gp41 probe can engage either a CXCR4 or a CCR5 cell-surface receptor on the T-Helper cell in order to open up an access portal for HIV to insert its genome into a T-Helper cell. It is well documented in the medical literature that a genetic defect leading to an abnormality in the CXCR4 cell-surface receptor prevents HIV virions from opening an access portal and inserting its genetic payload into such T-Helper cells. This genetic defect offers the subset of people carrying this genetic defect resistance to HIV infection. This example demonstrates the need for at least two types of glycoprotein probes to be present on the surface of a viral vector in order for a viral vector to be capable of opening an access portal and delivering the payload the vector carries into its host cell or target cell.

A delivery system that offered a defined means of targeting specific types of cells would invoke minimal or no response by the innate immune system when present in the body, and a delivery system that would be capable of inserting into cells a wide variety of differing payloads would significantly improve the current medical treatment options available to clinicians treating patients.

The solution to arriving at a versatile, workable delivery system that will meet the needs of a number of medical treatments involves three important elements. These elements include:

(1) configurable glycoprotein probes whereby more than one type of glycoprotein probe is to be used to engage and access specific target cell types in order to successfully deliver a payload into a specific cell type,
(2) an exterior envelope comprised of a protein shell or a lipid layer expressing the least number of cell-surface markers, such as the use of a stem cell, to act as the host cell to manufacture the delivery devices,
(3) a configurable core in the vector to enable it to carry and deliver a wide variety of payloads including proteins, chemicals, enzymes, RNAs, DNAs, nutrients, and molecules such as oxygen.

Viruses are obligate parasites. Viruses simply represent a carrier of genetic material and by themselves viruses are unable to replicate or carry out any form of biologic function outside their host cell. A ‘virion’ refers to the physical structure of a single complete virus as it exists outside of the host cell. Viruses are generally comprised of one or more nested shells constructed of one or more layers of protein, some with a lipid outer envelope, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biologic machinery for the virus to successfully replicate itself. Once the virus's genome is inside the host cell, the viral genome takes command of the cell's production machinery and causes the host cell to generate copies of the virus. As the viral copies exit the host cell, these virions set off in search of other host cells to infect.

Naturally occurring viruses exist in a number of differing shapes. The shape of a virus may be rod or filament like, icosahedral, or complex structures combining filament and polygonal shapes. Viruses generally have their outer wall comprised of a protein coat or an envelope comprised of lipids.

An outer envelope comprised of lipids may be in the form of one or two phospholipid layers. When the outer envelope is comprised of two phospholipid layers this is termed a lipid bilayer. A phospholipid is a composite molecule comprised of a polar or hydrophilic region on one end and a nonpolar or hydrophobic region on the opposite end. A lipid bilayer covering a virus, like the membrane of a cell, is constructed with the hydrophilic region of one of the phospholipid layers pointed toward the exterior of the virion and the hydrophilic region of the second phospholipid layer pointed inward toward the center of the virus virion; with the hydrophobic regions of each of the two lipid layers pointed toward each other. The outer envelope of some forms of virus may be comprised of an outer lipid layer or lipid bilayer affixed to a protein matrix for support, the protein matrix being located closer to the center of the virus virion than the lipid layer or lipid bilayer.

Spherical viruses are generally spherical in shape and may be comprised of an outer envelope and one inner shell or an outer envelope and multiple inner shells. Inner shells are approximately spherical in shape; this is because the proteins comprising the protein matrix shell have an irregular shape to their structure. In the case of a spherical virus with an outer envelope and one inner shell, the inner shell is often referred to as a nucleocapsid shell comprised of numerous capsid proteins attached to each other. In the case of a spherical virus being comprised of an outer envelope and multiple inner shells, the outermost inner viral shells may be referred to as comprised of a quantity of matrix proteins, where the innermost shell is referred to as a nucleocapsid and is comprised of a quantity of capsid proteins. The inner protein shells are nested inside each other.

Viruses carry genetic material in the form of deoxyribonucleic acid (DNA) or ribonucleic acid (RNA) in their nucleocapsid often referred to as the core or capsid. A virus is therefore generally considered to be a DNA virus if its genome is comprised of DNA or the virus is considered a RNA virus if its genome is comprised of RNA. Viruses may also carry enzymes as part of their payload. An enzyme such as ‘reverse transcriptase’ transforms a RNA viral genome into DNA. Protease enzymes modify the viral genome once it has entered a host cell. An integrase enzyme assists a DNA viral genome with insertion into the host cell's nuclear DNA. The payload is carried inside the virus's nucleocapsid shell.

The probes attached to the exterior of a virus are constructed to engage specific cell-surface receptors on specific cell types in the body. Only a cell that expresses cell-surface receptors that are capable of being engaged by the probes of a specific virus can act as a host for the virus. Viruses often use two probes to access a host cell. The first probe makes an initial attachment to the host cell, while the action of the virus's second probe often in conjunction with the action of the first probe cause an access portal to be created in the host cell's exterior plasma membrane. Once an access portal is formed, the virus inserts the contents of its payload into the host cell. Once the virus's genome is inside the cytoplasm of the host cell, any enzymes that accompanied the viral genome into the cell, may begin to modify or assist the virus's genome with infecting and taking control of the host cell's biologic functions.

Probes are attached to the exterior envelope of a virus virion. Probes may be in the form of a protein structure or may be in the form of a glycoprotein molecule. For viruses constructed with a protein matrix as its outer envelope, the probes tend to be protein structures. A portion of the protein structure probe is fixed or anchored in the protein matrix, while a portion of the protein structure probe extends out and away from the protein matrix. The portion of the protein structure probe extending out away from the virus virion is referred to as the ‘exterior domain’, the portion anchored in the protein matrix is the ‘transcending domain’. Some protein probes have a third segment that extends through the envelope and exists inside the virus virion, which is referred to as the ‘interior domain’. The exterior domain of a protein structure probe is intended to engage a specific cell-surface receptor on a biologically active cell the virus is targeting as its host cell.

Viruses that utilize a lipid layer as the outer envelope, are constructed with probes that tend to be glycoproteins. A glycoprotein is comprised of a protein segment and a carbohydrate segment. The carbohydrate segment of the glycoprotein molecule is fixed or anchored in the lipid layer of the outer envelope, while the protein segment extends outward and away from the outer envelope. The protein portion of a glycoprotein probe that extends outward and away from the outer envelope of a virus virion is intended to engage a cell-surface receptor on a biologically active cell the virus is targeting as its host cell.

Some forms of viruses that utilize a lipid layer as its envelope use protein structure probes. In this case, the portion of the protein structure probe that extends outward and away from the outer envelope is the ‘exterior domain’, the portion that is anchored in the lipid layer is the ‘transcending domain’ and again some protein structure probes have an ‘interior domain’ that exist inside the virion, which may also help anchor the protein structure probe to the virion. The exterior domain of a protein structure probe that extends outward and away from the outer envelope of a virus virion is intended to engage a cell-surface receptor on a biologically active cell the virus is targeting as its host cell.

When a virus carries a DNA payload and the viral DNA is inserted into the host cell, the viral DNA travels to the host cell's nucleus and is known to become inserted into the host cell's native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of the viral RNA by having the viral RNA converted to DNA. Once the viral RNA has been converted to viral DNA, the viral DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA. RNA viruses that bypass the host cell's DNA, cause the cell in general to generate copies of the necessary parts of the virus directly from the virus's RNA genome.

The human immunodeficiency virus (HIV) has an outer envelope comprised of a lipid bilayer. The lipid bilayer covers a protein matrix consisting of p17^{gag} proteins. Inside the p17^{gag} protein matrix is nested a nucleocapsid comprised of p24^{gag} proteins. Inside the nucleocapsid HIV carries its payload. HIV's genetic payload consists of two single strands of RNA. In addition to the two strands of HIV RNA, there are proteins that are carried in the core of the nucleocapsid along with the two RNA strands. These proteins include ‘reverse transcriptase’, ‘integrase’ and ‘protease’ molecules.

The T-Helper cell acts as HIV's host cell. HIV locates its host by utilizing at least two different types of probes located on its envelope. The HIV virion utilizes two types of glycoprotein probes affixed to the outer surface of its exterior envelope to engage a T-Helper cell. HIV utilizes a glycoprotein probe 120 to locate a CD4 cell-surface receptor on a T-Helper cell. Once an HIV glycoprotein 120 probe has successfully engaged a CD4 cell-surface receptor on a T-Helper cell a conformational change occurs in the probe and a glycoprotein 41 probe is exposed. The glycoprotein 41 probe's intent is to engage a CXCR4 or CCR5 cell-surface receptor on the same T-Helper cell. Once a glycoprotein 41 probe on the HIV virion successfully engages a CXCR4 or CCR5 cell-surface receptor, the HIV virion opens an access portal through the T-Helper cell's outer membrane.

Once the HIV virion has opened an access portal through the T-Helper cell's outer plasma membrane, the HIV virion inserts the two positive strand RNA molecules it carries into the T-Helper cell. Each RNA strand is approximately 9500 nucleotides in length. Inserted along with the RNA strands are the enzymes reverse transcriptase, protease and integrase. Once the virus's genome gains access to the interior of the T-Helper cell, the pair of RNA molecules are transformed in the cytoplasm to deoxyribonucleic acid by the reverse transcriptase enzyme. Following modification of the virus's genome to DNA, the virus's genetic information migrates to the host cell's nucleus. In the nucleus, with the assistance of the integrase protein, HIV's DNA becomes inserted into the T-Helper cell's native DNA. When the timing is appropriate, the now integrated viral DNA is decoded by the host cell's polymerase molecules and the virus's genetic information commands certain cell functions to carry out the replication process to construct copies of the human deficiency virus.

The outer layer of the HIV virion is comprised of a portion of the T-Helper cell's outer cell membrane. In the final stage of the replication process, as a copy of the HIV capsid, carrying the HIV genome, buds through the host cell's cell membrane the capsid acquires as its exterior envelope, a wrapping of lipid bilayer from the host cell's cell membrane. In the case of HIV, since the surface of the pathogen is covered by an envelope comprised of lipid bilayer taken from the host T-Helper cells, this feature allows the HIV virion the capacity to eluded the immune systems, since the cells comprising the immune system may find it difficult to tell the difference between the surface of an infectious HIV virion and the surface characteristics of a noninfected T-Helper cell.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C viral genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus life-cycle demonstrates that copies of a virus virion can be generated by inserting RNA into a host cell that functions as messenger RNA in the host cell. The Hepatitis C viral RNA genome functions as messenger RNA, acting as the template in conjunction with the biologic machinery of a host cell to produce the components that comprise copies of the Hepatitis C virion and the Hepatitis C viral RNA provides the biologic instructions to assemble the components into complete copies of the Hepatitis C virions. The Hepatitis C virus life-cycle clearly demonstrates that viral virions can be manufactured by a host cell without involving the nucleus of the cell.

Deciphering the existence, replication and behavior of viruses provides clear examples of several fundamental concepts, which include: (1) Viruses target specific cells in the body by means of identifying and engaging such target cells utilizing the probes projecting outward from the virus's exterior shell to make contact with cell-surface receptors located on the surface of the target cells, and (2) Viruses are capable of carrying various types of payloads including DNA, RNA and a variety of proteins.

Current medical therapy generally involves the administration of medications or biologic agents that tend to circulate the body by way of the blood stream and are capable of interacting with nearly all cells comprising the body that make contact with the circulation system. Adverse side effects occur when cells react to the toxic effects of medications or biologic agents. Often unwanted side effects occur due to cell types that are not targeted directly by the medications or biologic agents, being harmed by the presence of the medication or biologic agent circulating in the blood stream or tissues.

Current gene therapy approach to attempting to deliver a payload to cells in the body use modified forms of existing viruses to act as transport devices to deliver genetic information. This approach is severely limited by restricting the virus virion to target only cells the viral vector naturally seeks out and infects. Current gene therapy approach is further limited by using the pre-existing size of naturally occurring viruses, rather than being able to modify the size of the structure to be able to tailor the volumetric carrying capacity of the payload portion of the modified virus. Further gene therapy is restricted to utilizing naturally occurring viruses to deliver only genetic information; it has not previously been appreciated by those skilled in the art that virus-like transport devices might deliver to variety of specific cell types a wide variety of differing payloads as needed to successfully accomplish a specific medical treatment.

A dramatic treatment strategy, not previously recognized by those expert in the art, is the development of a transport vehicle that can be fashioned to seek out specific types of cells and deliver to these cells a variety of predetermined payloads as needed to successfully accomplish a specific medical treatment. Transport devices should be versatile enough to deliver a variety of payloads including DNA material, RNA material of various forms, simple proteins, complex proteins, conjugated proteins, globular proteins, polypeptides, chemicals, sugars, nutrients and elements such as oxygen. The RNA payloads include messenger RNA molecules, ribosomal RNA molecules, genetic RNA molecules intended to be converted to DNA, transport RNA molecules, small nuclear RNA molecules. The exterior envelope of a transport should be constructed so as not to alert the immune system of its presence to prevent rejection of the vehicles. Transport vehicles should be capable of being configured to target any specific cell type and engage and deliver their payload only to that specific predetermined cell type. To this point, such device has not been described in the literature.

### BRIEF SUMMARY OF THE INVENTION

Utilization of configurable microscopic medical payload delivery devices is meant to dramatically improve the efficiency and efficacy of medical care. Each configurable microscopic medical payload delivery device (CMMPDD) is intended to deliver specific medications or deliver specific biologic tool directly to specifically targeted cells in the body. By selecting the types of probes that are present on the surface of the configurable microscopic medical payload delivery devices, specific types of cells can be targeted. By delivering specific medications or biologic tools directly to specifically targeted cell types, the efficacy of the medication or the biologic tool is to be significantly improved. By delivering specific medications or biologic tools directly to specifically targeted cell types a reduction in side effects is appreciated due to the cells that are not intended to be exposed to the drug or a biologic tool that the configurable microscopic medical payload delivery devices are carrying, are spared exposure to potential toxic effects of drugs or biologic tools and therefore are not subject to harm.

### DETAILED DESCRIPTION

The future of medical treatment is the widespread utilization of configurable microscopic medical payload delivery devices (CMMPDD) to deliver medications or biologic tools directly to targeted cell types in the body.

For purposes of this text a medication includes chemical molecule, elements such as oxygen, sugars such as glucose, and other nutrients, which when purposely delivered to cells in the body produces a beneficial medical effect.

For purposes of this text a ‘biologic tool’ is a segment of DNA, a segment of RNA molecule, or a protein molecule such as an enzyme.

For purposes of this text an ‘external envelope’ refers to the outermost covering of a virus or a virus-like transport device or a configurable microscopic medical payload delivery device. The external envelope may be comprised of a lipid layer, a lipid bilayer, the combination of a lipid layer affixed to a protein matrix or the combination of a lipid bilayer affixed to a protein matrix.

For purposes of this text an ‘internal shell’ refers to a protein matrix shell nested inside the external envelope. The inner most protein matrix shell is termed the nucleocapsid. The proteins that comprise the nucleocapsid are termed capsid proteins. In the center or core of the nucleocapsid is where the payload is carried.

For purposes of this text ‘external probes’ are molecular structures that are utilized to locate and engage cell-surface receptors on biologically active cells. External probes are generally comprised of a portion which is anchored or fixed in the external envelope and a second portion that extends out and away from the external envelope. External probes may be comprised solely of a protein structure or an external probe may be a glycoprotein molecule.

For purposes of this text ‘glycoprotein molecule’ refers to a molecule comprised of a carbohydrate region and a protein region. Glycoprotein molecules that act as probes are generally anchored or fixed to a lipid layer utilizing the carbohydrate portion of the molecule as an anchor. The protein portion of the glycoprotein molecule which extends outward and away from the exterior envelope the glycoprotein has been affixed such that the protein region may function as a probe to locate and attach to the cell-surface receptor it was created to engage.

The concept of configurable microscopic medical payload delivery devices is modeled after naturally existing viruses. Configurable microscopic medical payload delivery devices in general are spherical in shape; though other shapes may be used as function might warrant the use of a particular shape. The spherical configurable microscopic medical payload delivery devices are comprised of an exterior envelope and one or more inner nested protein shells. A quantity of exterior protein structure probes and/or glycoprotein probes are anchored in the exterior lipid envelope and extend out and away from the exterior lipid envelope. Nesting of protein shells refers to progressively smaller diameter shells fitting snugly inside protein shells of a larger diameter. Inside the inner most protein shell, referred to as the nucleocapsid, is a cavity referred to as the core of the device. The core of the device is the space where the medically therapeutic payload the device carries is located.

Configurable microscopic medical payload delivery devices are generated to target certain specific cell types in the body. Configurable microscopic medical payload delivery devices target specific cell types by the configuration of probes affixed to the exterior envelope of the CMMPDD. By affixing specific probes to the exterior envelope of the CMMPDD, these probes intended to engage and attach only to specific cell-surface receptors located on certain cell types in the body, the CMMPDD will deliver its payload only to those cell types that express compatible and engagable specific cell-surface receptors. In a similar fashion where the exterior probes of a naturally occurring virus engage specific cell-surface receptors present on the surface of the virus's host cell and only the designated host cell, the CMMPDD's exterior probes are configured to engage cell-surface receptors on a specific type of target cell. In this manner, the payload of medication or biologic tools carried by CMMPDD will be delivered only to specific types of cells in the body. The exterior probes on the surface of a CMMPDD will vary as needed so as to effect the CMMPDD delivery of payloads to cell types as needed to effect a medical treatment.

The size of configurable microscopic medical payload delivery devices is to depend upon the volume size of the payload the CMMPDD is required to carry and deliver to a target cell. The size of a CMMPDD is dependent upon the diameter of the inner protein matrix shells. The diameter of each inner protein matrix shell is governed by the number of protein molecules utilized to construct the protein matrix shell at the time the protein matrix shell is generated. Increasing the number of proteins that comprise a protein matrix shell, increases the diameter of the protein matrix shell. The external lipid envelope wraps around and covers the outermost protein matrix shell. The larger the volume of the core of the CMMPDD, the greater the physical size payload the CMMPDD is able to carry. The size of the configurable microscopic medical payload delivery device is to be the size of cell (approximately 10⁻⁴ m in diameter) or less, generally detectable by a light microscope or, as needed, an electron microscope. The size of the CMMPDD is not to be too large such that it would generate a burden to the body by damaging organ tissues through clogging blood vessels, and the maintaining a small enough size that the CMMPDD can be properly disposed of by the body once the CMMPDD has delivered its payload to its target cell. The dimensions of each type of CMMPDD are to be tailored to the mission of the CMMPDD, which takes into account the type of target cell, the size of the payload that is to be delivered to the target cells and the length of time the CMMPDD may engage the target cell.

Being enveloped in an external lipid layer, configurable microscopic medical payload delivery devices possess the advantage of having their exterior appear similar to the plasma membrane that acts as an outside covering for the cells that comprise the body. By appearing similar to existing plasma membranes, the CMMPDDs appear similar to naturally occurring structures found in the body, affording the CMMPDD the capability to avoid detection by a body's immune system because the exterior of the CMMPDD mimics the cells comprising the body and the surveillance cells of the immune system find it difficult to discern between the CMMPDD and naturally occurring cells comprising the body.

To carry out the process of manufacturing a configurable microscopic medical payload delivery device, a primitive cell such as a stem cell is selected. The reason for utilizing primitive cells such as stems cells as the host cell, is that the CMMPDD acquires its outer envelope from the host cell and the more primitive the host cell, the fewer in number the identifying protein markers are present on the surface of the CMMPDD. The fewer the identifying surface proteins present on the outer envelope of the CMMPDD, the less likely a body's immune system will identify the CMMPDD as an invader and therefore less likely the body's immune system will react to the presence of the CMMPDD and reject the CMMPDD by attacking and neutralizing the CMMPDD.

Stem cells used as host cells to manufacture quantities of CMMPDD product are selected per histocompatibility markers present on their surface. Certain histocompatibility markers present on the surface of the final CMMPDD product will be less likely to cause a reaction in a specific patient based on the genetic profile of the patient's histocompatibility markers. A similar histocompatibility match is done when donor organs are selected to be given to recipients to avoid rejection of the donor organ by the recipient's immune system.

The selected stem cell used to manufacture configurable microscopic medical payload delivery devices goes through several steps of maturation before it is capable of generating therapeutic CMMPDD product. Messenger RNA would be inserted into the host stem cell that would code for the general physical outer structures of the CMMPDD. Messenger RNA would be inserted into the host that would generate surface probes that would target the surface receptors on specifically target cells. Messenger RNA would be inserted into the host that would be used to generate the therapeutic payload. Similar to how copies of a naturally occurring virus are produced, assembled and released from a host cell, copies of the CMMPDD would be produced, assembled and released from a host cell. Once released from the host cell, the copies of the CMMPDD would be collected, then pooled together to produce a therapeutic dose that would result in a medically beneficial effect.

The construction of the configurable microscopic medical payload delivery devices is performed by taking stem cells and inserting modified viral genetic programming into the stem cells. Stem cells are chosen as the host cell due to the low number of surface markers, which leads to less antigenicity in configurable microscopic medical payload delivery devices when the configurable microscopic medical payload delivery devices are released by the host cells and wrapped in an outer envelope comprised of the host cells' plasma membrane.

The stem cells used as host cells are suspended in a broth of nutrients and are kept at an optimum temperature to govern the rate of production of the CMMPDD product. Similar to the natural production of the Hepatitis C virus, the configurable microscopic medical payload delivery devices ‘production genome’ is introduced into the host stem cells. The configurable microscopic medical payload delivery devices production genome carries genetic instructions to cause the host cells to manufacture the configurable microscopic medical payload delivery devices' outer protein wall, the inner protein matrixes, the surface probes the configurable microscopic medical payload delivery device is to have affixed to its outer envelope and the payload the configurable microscopic medical payload delivery devices are to carry; and the instructions to assemble the various pieces into the final form of the configurable microscopic medical payload delivery devices and the instructions to activate the budding process. The resultant configurable microscopic medical payload delivery devices are collected from the nutrient broth surrounding the host cells and placed together into doses to be used as a treatment for a medical disease.

The ‘production genome’ are an array of messenger RNAs that are directly translated by the host cell's internal enzymes. The production genome dictates the characteristics of the final version of the CMMPDD that buds from the host stem cell and is released and is to be utilized as a medical treatment. The production genome is specifically tailored to code for the surface probes that will seek and engage a specific type of target cell. The production genome also carries the instructions to code for the production of the type of payload to be delivered to the specific type of target cell. In the case of a nutrient, the production genome carries the instructions to place a proper amount of nutrient into the CMMPDD. The ‘production genome’ varies depending upon the configuration of the CMMPDD to effect a specific medical treatment.

The configurable microscopic medical payload delivery device represents a very versatile delivery device. There are an estimated 100,000 genes located in the human genome. CMMPDD could be used to deliver any of the 100,000 genes to any specific cell type in the body. Regarding RNAs, CMMPDD could be utilized to deliver to specific cell types messenger RNAs, ribosomal RNAs, transport RNAs, small nuclear RNAs. With regards to messenger RNAs, there are at least 210 different cell types in the human body and there are at least 30,000 different proteins that the human body produces. Each of the 30,000 proteins are generated by a cell translating one or more mRNAs responsible for production of a particular protein. The number of different medical treatment options that are possible as a result of delivering messenger RNAs to specific cell types is approximately 6,000,000. A wide variety of proteins could be transported to specific cell types by means of CMMPDD. There are numerous presently existing medications and numerous emerging medications that could be delivered directly to specific cell types per the transport capability of CMMPDD. There are a wide variety of nutrients that could be delivered to specific cells by means of CMMPDD.

As an example of this method, to treat diabetes mellitus utilizing configurable microscopic medical payload delivery devices to deliver to Beta cells messenger RNA coded to produce insulin, the following production process is followed in the lab: (1) human stem cells are selected. (2) Into the selected stem cells is placed the production genome constructed, in this case, specifically as a means to treat diabetes mellitus. The RNA production genome contains genetic instructions to cause the host stem cells to manufacture the configurable microscopic medical payload delivery devices' outer protein wall, the inner protein matrix, surface probes to include glycoprotein probes that engage the GPR40 cell-surface receptor present on the surface of Beta cells located in the Islets of Langerhans in the pancreas, and the payload, in this case messenger RNA constructed to effect the production of the insulin molecule; and the biologic instructions to assemble the components into the final form of the configurable microscopic medical payload delivery devices; and the biologic instructions to activate the budding process. (3) Upon insertion of the RNA production genome dedicated to producing a messenger RNA coded to produce insulin, into the host stem cells, host stem cells respond by (i) simultaneously translating the different segments of the RNA production genome to produce the proteins that comprise the exterior protein wall, the inner protein matrix molecules, the surface probes to seek out and engage Beta cells, the mRNA payload to produce insulin, and (ii) decoding the RNA instructions to assemble the components into the configurable microscopic medical payload delivery devices. (4) Upon assembly, the configurable microscopic medical payload delivery devices bud through the cell membrane of the host stem cell. (5) At the time of the budding process, the configurable microscopic medical payload delivery devices acquire an outside envelope wrapped over the outer protein shell, this outer envelope comprised of a portion of the plasma membrane from the host stem cell as the configurable microscopic medical payload delivery devices exit the host cell. (6) The resultant configurable microscopic medical payload delivery devices are collected from the nutrient broth surrounding the host stem cells. (7) The configurable microscopic medical payload delivery devices are washed in sterile solvent to remove contaminants. (8) The configurable microscopic medical payload delivery devices are removed from the sterile solvent and suspended in a hypoallergenic liquid medium. (9) The configurable microscopic medical payload delivery devices are separated into individual quantities to facilitate storage and delivery to physicians and patients. (10) The configurable microscopic medical payload delivery devices transported in the hypoallergenic liquid medium is administered to a diabetic patient per injection in a dose that is tailored to receiving patient's requirement to produce sufficient amount of insulin to control the blood sugar. (11) Upon being injected into the body, the configurable microscopic medical payload delivery devices migrate to the Beta cells located in the Islets of Langerhans by means of the patient's blood stream. (12) Upon the configurable microscopic medical payload delivery devices reaching the Beta cells, the configurable microscopic medical payload delivery devices engage the cell-surface receptors located on the Beta cells and insert the payload they carry into the Beta cells. The payload, in this case being messenger RNA coded to produce insulin, is translated by the cell's ribosomes to produce insulin molecules. The increase in insulin production by Beta cells successfully manages diabetes mellitus.

### CONCLUSIONS, RAMIFICATION, AND SCOPE

Accordingly, the reader will see that the configurable microscopic medical payload delivery device to deliver medically therapeutic payloads to specifically targeted cell types provides advantages over existing art by being a delivery device that (1) is constructed to seek out and engage specific types of cells by design based on medical need, (2) is versatile enough in its construction to deliver a wide variety of possible payloads to specific cell types, and (3) is constructed with a surface envelope that will avoid detection by the innate immune system so as not to activate the immune system to its presence; for these reasons this represents a new and unique medical delivery device that has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

What is claimed:

1. A configurable microscopic medical payload delivery device comprised of:
(a) an exterior envelope,
(b) a quantity of interior shells, and
(c) a quantity of configurable exterior probes attached in a manner a segment of said exterior probes is to project out and away from said exterior envelope, while a segment of said exterior probes is embedded in said exterior envelope,
whereby said configurable microscopic medical payload delivery device is intended to deliver a medically therapeutic payload to a specific cell type in order to produce a specific medically beneficial effect,
whereby said exterior probes are intended to engage specific cell-surface receptors on said specific cell type,
whereby said interior shells are versatile enough in their construction to carry within the nucleocapsid a wide variety of possible payloads to said specific cell types.

2. The configurable microscopic medical payload delivery device in claim 1 wherein said external envelope is selected from the group consisting of a lipid layer, a lipid bilayer, a protein matrix, a lipid layer affixed to a protein matrix, and a lipid bilayer affixed to a protein matrix.

3. The configurable microscopic medical payload delivery device in claim 1 wherein said quantity of internal shells are comprised of a quantity of nested sphere-like concentric protein matrix structures.

4. The configurable microscopic medical payload delivery device in claim 1 wherein said exterior probes are comprised of a quantity of protein molecules.

5. The protein molecules in claim 4 wherein said protein molecules are comprised of a segment of said protein molecule which extends outward and away from said exterior envelope, attached to a segment of said protein molecule which is embedded in said exterior envelope to hold said protein molecule affixed to said exterior envelope,
whereby said segment of said protein molecules which extends outward and away from said exterior envelope is intended to engage said specific cell-surface receptors on said specific cell type.

6. The protein molecules in claim 4 wherein said protein molecules are comprised of a plurality of protein molecules,
whereby, at least two differing configurations of said protein molecules may be needed to successfully engage said specific cell type with one type of configuration of said protein molecule engaging one type of said specific cell-surface receptor, while a differing type of configuration of said protein molecule is required to engage a differing type of said specific cell-surface receptor in order for said configurable microscopic medical payload delivery device to insert said medically therapeutic payload said configurable microscopic medical payload delivery device carries into intended said specific cell type.

7. The configurable microscopic medical payload delivery device in claim 1 wherein said exterior probes are comprised of a quantity of glycoprotein molecules.

8. The glycoprotein molecules in claim 7 wherein said glycoprotein molecules are comprised of a protein segment which extends outward and away from said exterior envelope, which is attached to a carbohydrate segment, said carbohydrate segment being embedded in said exterior envelope to hold said glycoprotein molecule affixed to said exterior envelope,
whereby said protein segment which extends outward and away from said exterior envelope is intended to engage said specific cell-surface receptor on said specific cell type.

9. The glycoprotein molecules in claim 7 wherein said glycoprotein molecules are comprised of a plurality of glycoprotein molecules,
whereby, at least two differing configurations of said glycoprotein molecules may be needed to successfully engage said specific cell type with one type of configuration of said glycoprotein molecule engaging one type of said specific cell-surface receptor, while a differing type of configuration of said glycoprotein molecule is required to engage a differing type of said specific cell-surface receptor in order for said configurable microscopic medical payload delivery device to insert said medically therapeutic payload into said specific cell type.

10. A configurable medical treatment payload delivery means for inserting medically beneficial payloads into predetermined biologically active cells of a particular type comprising:
(a) an exterior envelope,
(b) a quantity of interior shells, and
(c) a quantity of exterior probes attached to said exterior envelope,
whereby said configurable medical treatment payload delivery means is intended to deliver medically therapeutic payloads to said predetermined biologically active cells of a particular type in order to produce a medically beneficial effect,
whereby said exterior probes are intended to engage specific cell-surface receptors on said predetermined biologically active cells of a particular type,
whereby said interior shells are versatile enough in their construction to carry within the nucleocapsid a wide variety of possible payloads to said predetermined biologically active cells of a particular type.

11. The configurable medical treatment payload delivery means in claim 10 wherein said external envelope is selected from the group consisting of a lipid layer, a lipid bilayer, a protein matrix, a lipid layer affixed to a protein matrix, and a lipid bilayer affixed to a protein matrix.

12. The configurable medical treatment payload delivery means in claim 10 wherein said internal shell is comprised of a quantity of nested concentric protein matrix sphere-like structures.

13. The configurable medical treatment payload delivery means in claim 10 wherein said exterior probes are comprised of a quantity of protein molecules.

14. The protein molecules in claim 13 wherein said protein molecules are comprised of a segment of said protein molecule which extends outward and away from said exterior envelope, which is attached to a segment of said protein molecule embedded in said exterior envelope to hold said protein molecule affixed to said exterior envelope,
whereby said segment of said protein molecule which extends outward and away from said exterior envelope is intended to engage said specific cell-surface receptor on said predetermined biologically active cells of a particular type.

15. The protein molecules in claim 13 wherein said protein molecules are comprised of a plurality of protein molecules,
whereby, at least two differing configurations of said protein molecules may be needed to successfully engage said predetermined biologically active cells of a particular type with one type of configuration of said protein molecule engaging a type of said specific cell-surface receptor, while a differing type of configuration of said protein molecule is required to engage a differing type of said specific cell-surface receptor in order for said configurable medical treatment payload delivery means to insert said medically therapeutic payload into intended said predetermined biologically active cells of a particular type.

16. The configurable medical treatment payload delivery means in claim 10 wherein said external probes are comprised of a quantity of glycoprotein molecules.

17. The glycoprotein molecules in claim 16 wherein said glycoprotein molecules are comprised of a protein segment which extends outward and away from said exterior envelope, which is attached to a carbohydrate segment, said carbohydrate segment intended to be embedded in said exterior envelope to hold said glycoprotein molecule affixed to said exterior envelope,
whereby said protein segment which extends outward and away from said exterior envelope is intended to engage said specific cell-surface receptor on said predetermined biologically active cells of a particular type.

18. The glycoprotein molecules in claim 16 wherein said glycoprotein molecules are comprised of a plurality of glycoprotein molecules,
whereby, at least two differing configurations of said glycoprotein molecules may be needed to successfully engage said predetermined biologically active cells of a particular type with one type of configuration of said glycoprotein molecule engaging a type of said specific cell-surface receptor, while a differing type of configuration of said glycoprotein molecule is required to engage a differing type of said specific cell-surface receptor in order for said configurable medical treatment payload delivery means to insert said payload into said predetermined biologically active cells of a particular type.
